(19) United States
(12) Patent Application Publication
(10) Pub. No.: US 2025/0152525 A1
(43) Pub. Date: May 15, 2025

(54) COMPOSITIONS AND METHODS FOR THE TREATMENT OF TESTOSTERONE DEFICIENCY IN MALES

(71) Applicant: Woody's Medical LLC, Scottsdale, AZ (US)

(72) Inventors: Austin Lucht, Scottsdale, AZ (US); Suzanne Bentz, Scottsdale, AZ (US)

(73) Assignee: Woody's Medical LLC, Scottsdale, AZ (US)

(21) Appl. No.: 18/505,241

(22) Filed: Nov. 9, 2023

(51) Int. Cl. A61K 31/138; A61K 9/48; A61K 31/40; A61K 31/4535; A61K 31/55

(52) U.S. Cl. CPC A61K 31/138; A61K 9/4866; A61K 31/40; A61K 31/4535; A61K 31/55

(57) ABSTRACT

In various embodiments compositions for the treatment of testosterone deficiency (TD, or “low-T”) and/or secondary hypogonadotropic hypogonadism comprise at least one selective estrogen receptor modulator (SERM) and a carrier, wherein the composition is entirely devoid of any estrogenically active substances capable of lowering testosterone levels. In preferred embodiments, compositions comprise only the (E)-stereoisomer of clomiphene and are entirely devoid of the estrogenically active (Z)-stereoisomer of clomiphene. In various examples, compositions are enclosed in a rapidly disintegrating capsule configured for immediate release. In preferred embodiments, the carrier comprises microcrystalline cellulose that functions as both bulk filler and disintegrant.

### FIELD

This disclosure generally relates to pharmaceutical compositions, and more specifically, to orally administered compositions usable to treat testosterone deficiency in males.

### BACKGROUND

Men that clinically present with low testosterone will likely be diagnosed with an underlying cause such as, for example, hypogonadism, a condition where the testicles produce little to no testosterone, testicular injury, infection, cancer (or other chronic diseases), obesity, alcohol abuse, stress, diminished thyroid function, or recent exposure to chemotherapy. In certain cases, testosterone deficiency (TD) is a result of secondary hypogonadotropic hypogonadism, which is an inadequacy in the hypothalamic-pituitary-gonadal axis. Symptoms of testosterone deficiency (TD) include reduced sex drive, erectile dysfunction, loss of body hair, reduced beard growth, muscle atrophy, fatigue, and depression.

Treatment of TD include testosterone replacement therapy, such as delivery of the hormone itself through skin patches, topical gels, oral tablets, implanted pellets, and hormone injections.

In addition to testosterone replacement therapy, TD caused by secondary hypogonadotropic hypogonadism has been treated by certain non-steroidal selective estrogen receptor modulators (SERMs). A SERM compound acts by antagonizing estrogen receptors in the pituitary gland, interfering with the negative feedback by estrogen on the hypothalamic-pituitary-gonadal axis, resulting in increased gonadotropin secretion and increased gonadal production of testosterone. In men with secondary hypogonadotropic hypogonadism, this improves testosterone levels and sperm motility without adversely affecting fertility. The most common SERMs include tamoxifen, toremifene, raloxifene, lasofoxifene, bazedoxifene and clomifene citrate. See, for example, A. Rambhatla, “The Role of Estrogen Modulators in Male Hypogonadism and Infertility,” Rev. Urol., 18(2), 66-72, (2016).

Nonetheless, various SERM compounds exist as mixtures of stereoisomers having polarizing opposite pharmacological effects, little of which has been understood. Further, various administration routes have proved ineffective and many side effects have resulted, including gynecomastia, weight gain and hair loss.

In view of these and other serious shortcomings, new dosage forms and treatment regimens are still needed for management of testosterone deficiency in male patients.

### SUMMARY

It has now been surprisingly discovered that certain SERMs, particularly enclomifene, the (E)-stereoisomer of clomifene, are more effective for treatment of testosterone deficiency when administered as needed in instant delivery (ID) capsules. This finding is contrary to prior teaching that SERMs like clomifene must be administered as slow release orally ingested tablets in a regimen prescribing chronic daily administration. SERMs like clomifene have not been previously compounded or dosed in the form of ID capsules. Most importantly, there has been no regard to the differences in pharmacological effect between enclomifene and zuclomiphene, the (E)- and (Z)-stereoisomers of clomifene, respectively.

In various embodiments, new compositions for the treatment of testosterone deficiency (or more simply, “TD compositions”) are described. In various embodiments, TD compositions are compounded in a dosage form for oral administration and rapid disintegration and release.

In various embodiments, new TD compositions comprise an immediate release oral dosage form.

In various embodiments, new TD compositions comprise an immediate release capsule oral dosage form.

In various embodiments, a pharmaceutical dosage form for oral administration of a TD composition comprises a capsule and a TD composition comprising at least one SERM and a filler, optionally an excipient, enclosed within the capsule.

In various embodiments, a method of treating testosterone deficiency (TD) is disclosed.

In various embodiments of the method, the individual in need thereof is diagnosed as having testosterone deficiency, such as through clinical measurement of testosterone in the individual or evaluated as having any one of the related indications of reduced sex drive, erectile dysfunction, loss of body hair, reduced beard growth, muscle atrophy, fatigue, or depression. In capsule form, compositions of the present disclosure find use in testosterone replacement therapy (TRT) which injections are not a viable option. Injectable TRT is not an option when males are wanting to conceive due to exogenous testosterone decreases spermatogenesis and motility, or if patients are not favorable to injections.

In various embodiments, compositions according to the present disclosure also find use in the treatment of testicular atrophy, such as caused by injectable testosterone replacement therapy (TRT). In certain examples, compositions of the present disclosure can be administered several times per week to normalize testicular size.

In various embodiments, a pharmaceutical composition for treatment of testosterone deficiency (TD) and/or secondary hypogonadotropic hypogonadism comprises at least one selective estrogen receptor modulator (SERM); a carrier; and optionally, an excipient; wherein the composition is in the form of a loose dry powder amenable to packaging into individual dosage forms, and wherein the composition is entirely devoid of estrogenically active substances.

In various embodiments, the selective estrogen receptor modulator (SERM) is selected from the group consisting of tamoxifen, toremifene, raloxifene, lasofoxifene, bazedoxifene, enclomifene, pharmaceutically acceptable salts thereof, and mixtures thereof.

In various embodiments, a pharmaceutical dosage form for oral administration comprises a capsule; and a composition comprising at least one selective estrogen receptor modulator (SERM); a carrier; and optionally, an excipient enclosed therein.

In various embodiments, the carrier comprises microcrystalline cellulose.

In various embodiments, the optional excipient is at least one of a disintegrant, a colorant, a flavoring agent, a sweetener, a preservative, and a stabilizer.

In various embodiments, a pharmaceutical composition for treatment of testosterone deficiency (TD) and/or secondary hypogonadotropic hypogonadism consists essentially of enclomifene ((E)-2-(4-(2-chloro-1,2-diphenylvinyl)phenoxy)-N,N-diethylethan-1-amine) and microcrystalline cellulose, wherein the pharmaceutical composition is entirely devoid of zuclomifene ((Z)-2-(4-(2-chloro-1,2-diphenylvinyl)phenoxy)-N,N-diethylethan-1-amine).

In various embodiments, a single dose pharmaceutical capsule consists essentially of from about 100 mg to about 300 mg of a composition consisting essentially of from about 15 wt. % to about 25 wt. % enclomifene citrate and from about 75 wt. % to about 85 wt. % microcrystalline cellulose, based on the total weight of the composition; and a capsule enclosing said composition, wherein said composition is entirely devoid of zuclomifene ((Z)-2-(4-(2-chloro-1,2-diphenylvinyl)phenoxy)-N,N-diethylethan-1-amine), or salts, solvates, hydrates, or prodrugs thereof.

In various embodiments, the capsule comprises gelatin or vegetarian hydroxypropyl methylcellulose (HPMC), and wherein the capsule is optionally laser-perforated with a plurality of holes for water ingress.

In various embodiments, a method of treating testosterone deficiency (TD) and/or secondary hypogonadotropic hypogonadism in a male individual comprises orally administering to the individual in need thereof a therapeutically effective amount of a composition comprising at least one selective estrogen receptor modulator (SERM); a carrier; and optionally, an excipient, wherein the composition is entirely devoid of estrogenically active substances.

In various embodiments, the testosterone deficiency (TD) and/or secondary hypogonadotropic hypogonadism is characterized by reduced sex drive, erectile dysfunction, loss of body hair, reduced beard growth, muscle atrophy, fatigue, or depression.

In various embodiments, the testosterone deficiency (TD) and/or secondary hypogonadotropic hypogonadism is characterized by testicular atrophy caused by injectable testosterone replacement therapy (TRT).

In various embodiments, the therapeutically effective amount comprises orally administering from about 3 mg to about 50 mg of the at least one SERM daily.

In various embodiments, the at least one SERM is enclomifene ((E)-2-(4-(2-chloro-1,2-diphenylvinyl)phenoxy)-N,N-diethylethan-1-amine) and the carrier is microcrystalline cellulose.

In various embodiments, the therapeutically effective amount comprises daily administration of a capsule enclosing said composition, wherein each capsule comprises from about 120 mg to about 150 mg of the composition, and wherein each capsule delivers from about 3 mg to about 50 mg enclomifene actives.

In various embodiments, each capsule preferably delivers about 3.125 mg, 6.25 mg, 12.5 mg, 25 mg or 50 mg enclomifene actives.

### DETAILED DESCRIPTION

The detailed description of exemplary embodiments refers to the accompanying drawings, which show exemplary embodiments by way of illustration and best mode. While these exemplary embodiments are described in enough detail to enable those skilled in the art to practice the invention, other embodiments may be realized, and logical, chemical, and mechanical changes may be made without departing from the spirit and scope of the inventions. Thus, the detailed description is presented for purposes of illustration only and not of limitation. For example, unless otherwise noted, the steps recited in any of the method or process descriptions may be executed in any order and are not necessarily limited to the order presented. Furthermore, any reference to singular includes plural embodiments, and any reference to more than one component or step may include a singular embodiment or step. Also, any reference to attached, fixed, connected or the like may include permanent, removable, temporary, partial, full and/or any other possible attachment option. Additionally, any reference to without contact (or similar phrases) may also include reduced contact or minimal contact.

Compositions for the treatment of testosterone deficiency (TD), comprising a selective estrogen receptor modulator (or SERM), are described. In various embodiments, a TD composition in accordance with the present disclosure comprises at least one SERM, a filler, and optionally at least one excipient. In various embodiments, a filler includes a disintegrant or it exhibits properties of a disintegrant while providing bulk volume and weight to a composition.

In certain examples, a composition for the treatment of testosterone deficiency (TD) comprises enclomifene, a filler, and optionally at least one excipient, wherein the composition is entirely devoid of zuclomifene.

### Definitions

As used herein, the term “testosterone deficiency” refers to a condition in a male individual manifesting as lower than normal levels of the hormone testosterone. Testosterone deficiency (TD), or the presence of lower than normal levels of testosterone in a male subject may be caused by secondary hypogonadotropic hypogonadism and may cause symptoms in the individual such as reduced sex drive, erectile dysfunction, loss of body hair, reduced beard growth, muscle atrophy, fatigue, or depression. For purposes herein, treatment of testosterone deficiency is interchangeable with treatment of secondary hypogonadotropic hypogonadism when the TD in the male subject at issue is caused by secondary hypogonadotropic hypogonadism.

As used herein, the acronym “SERM” refers to a “selective estrogen receptor modulator,” which is a class of estrogen receptor antagonists. A SERM antagonizes the estrogen receptors in the pituitary gland, disrupting the negative feedback loop by estrogen towards the hypothalamic-pituitary-gonadal axis, ultimately resulting in an increase in gonadotropin secretion and an increase in testosterone. SERMs are used in the treatment of breast cancer, osteoporosis and postmenopausal symptoms in women, and to treat persistent low testosterone in men as a result of secondary hypogonadotropic hypogonadism. SERMs are generally characterized as non-steroidal synthetic agents capable of binding to estrogen receptors to produce a change in the activity of the receptor.

As used herein, the terms “enclomifene” or “enclomiphene” or “(E)-clomifene” refer to the pharmaceutical active having the chemical structure according to Formula (I):

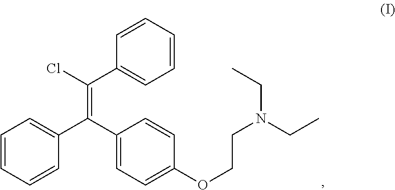 [SMILES: CCN(CC)CCOc1ccc(/C(=C(/Cl)c2ccccc2)c2ccccc2)cc1]

with IUPAC name (E)-2-(4-(2-chloro-1,2-diphenylvinyl)phenoxy)-N, N-diethylethan-1-amine, CAS No. 15690-57-0 and MW 405.97 g/mol. In various embodiments, enclomifene salts, solvates, hydrates, and prodrugs thereof may be used in various compositions herein, such as for example, enclomifene citrate (CAS No. 7599-79-3, MW 598.08 g/mol). Enclomifene is the (E)-stereoisomer of clomifene.

As used herein, the terms “zuclomifene” or “zuclomiphene” or “(Z)-clomifene” refer to the pharmaceutical active having the chemical structure according to Formula (II):

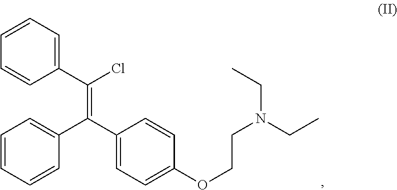 [SMILES: CCN(CC)CCOc1ccc(/C(=C(\Cl)c2ccccc2)c2ccccc2)cc1]

with IUPAC name (Z)-2-(4-(2-chloro-1,2-diphenylvinyl)phenoxy)-N, N-diethylethan-1-amine, CAS No. 15690-55-8 and MW 405.97 g/mol. In various embodiments, zuclomifene salts, solvates, hydrates, and prodrugs thereof may be of interest, such as for example, zuclomifene citrate. Zuclomifene is the (Z)-stereoisomer of clomifene.

As used herein, the terms “clomifene” or “clomiphene” refer to the mixture consisting of about 38 wt. % zuclomifene and 62 wt. % enclomifene. Zuclomifene is mildly estrogenic and antigonadotrophic, whereas enclomifene is antiestrogenic. Using clomifene in the treatment of TD is nonsensical in that the (E)- and (Z)-stereoisomers have opposite effects, most notably that (E)-clomifene (enclomifene) increases testosterone production whereas (Z)-clomifene (zuclomifene) reduces testosterone production. Therefore, it is undesirable to include zuclomifene in a composition for the treatment of testosterone deficiency (TD).

As used herein, the term “estrogenically active substances” refers to steroidal and non-steroidal compounds shown to be estrogenic and thus capable of activating estrogen receptors and consequently reducing testosterone levels in men. Such compounds include, but are not limited to, estradiol, 2-hydroxyestradiol, 4-hydroxyestradiol, 4-methyoxyestradiol, diethylstilbestrol, and zuclomifene. In preferred compositions herein for the treatment of testosterone deficiency, estrogenically active substances are avoided, meaning compositions herein are entirely devoid of estrogenically active substances that cause a reduction of testosterone levels in men.

As used herein, the term “immediate release” or “immediate delivery” or “instant delivery” or “ID” refer to a pharmaceutical dosage form designed to fall apart, dissolve, or otherwise disintegrate as quickly as possible. Typically, a dosage form for immediate release will be an oral dosage form, such as a tablet for sublingual drug administration or a capsule for swallowing with subsequent GI drug delivery. Capsule dosage forms may be designed for immediate drug release by physical or chemical changes to either the capsule shell, or the powder fill composition, or both. For example, hard capsule shells may be laser-drilled with holes that allow entry of water in the gut. In other examples, vegetarian cellulose capsules, such as hydroxypropyl methylcellulose (HPMC capsules), may be preferred over gelatin capsules in view that gelatin polymers tend to be crosslinked which reduces dissolution rates. In various examples, one or more disintegrating agents (called “disintegrants”) may be added into the powder fill composition of a capsule, or into the capsule shell itself to accelerate physical deterioration. In various embodiments, immediate release may also be coupled with increased GI absorption, and in those instances an intestinal permeation enhancer may be included in an immediate release composition so that there is not only rapid dissolution of the capsule but also intestinal absorption. In various embodiments, a standard gel cap or powder filled hard gelatin capsule may provide immediate release in the gut simply in response to physiological conditions of heat and acid in the stomach, sufficiently capable of dissolving a gelatin shell in due course. Immediate release tends to be the opposite of controlled release or sustained release.

As used herein, the term “carrier” refers to a material or mixture of materials that are not pharmacologically active, and that may be added in quantity sufficient to complete a formula to “100 wt. %” total. In various embodiments, TD compositions herein comprise at least one SERM and optional excipient, with the remainder comprising a carrier. Carriers increase weight and may be seen to convey pharmaceutical actives to a particular locale, but typically do not contribute to any pharmacological effect. Pharmaceutically acceptable carriers are known to the pharmaceutical arts, and include such substances as monosaccharides and disaccharides, (mannitol, lactose, dextrose), cellulosic substances (microcrystalline cellulose MCC), carbonates (calcium carbonate), phosphates, (calcium phosphate), and sulfates (calcium sulfate). For an exhaustive listing of pharmaceutically acceptable carriers, see “Handbook of Pharmaceutical Excipients, 6^{th} Edition, R. C Rowe, et al., editors, Pharmaceutical Press, London, 2009. Microcrystalline cellulose has properties of a disintegrant and thus can be used as both disintegrant and carrier. MCC enhances drug dissolution by expanding in size when in contact with water, causing tablets and capsules to break apart faster in the GI tract. In other embodiments, a TD composition herein may include a separate disintegrant as an excipient with a carrier.

As used herein, the term “excipient” refers to additional functional ingredients in a TD composition, and thus distinct from inert filler. Excipient refers to those ingredients that although functional, play a minor role in the composition and no pharmacological role. These ingredients typically include disintegrants, colors, flavorants, sweenteners, and preservatives.

As used herein, the term “composition” takes on the ordinary meaning in formulation chemistry as a combination of ingredients. In various embodiments, a composition is designed to adopt a particular physical form, or at least be amenable to physical change into a desired physical form, which may be the dosage form for a particular treatment regimen. Typically, a composition is made homogeneous by mixing or blending, although not all liquid compositions are colorless and transparent and not all powder compositions are white and perfectly powdered or granular. Compositions comprising an emulsion, dispersion or suspension may be homogeneous because the droplets or particles are evenly spread in a carrier. So, for example, a composition herein may be in the form of a thin liquid (having a viscosity at or near that of water), a viscous liquid (having a liquid of viscosity greater than water), a paste, a cream, a jelly, a gel, or a powder. Ingredients for a composition herein are generally shown “as added,” meaning there is a possibility for one or more chemical reactions between ingredients once the ingredients are mixed together, such as into a common carrier. One skilled in the art of formulation chemistry can recognize whether ingredients might react in a mixture. These reactions can include neutralization (e.g., between acid and alkali ingredients), mixed micelle formation (mixed surfactants in liquid systems) or other encapsulation phenomena, hydrolysis, and so forth. In various embodiments, a composition herein comprises a blended powder that can be packed into capsules for oral administration. In some instances, a composition may take into consideration an outer encasing when that material is also included in the administration of the composition to an individual. For example, a gelatin capsule may be included in the listing of ingredients for a composition, or perhaps just the ingredients of the contents of the capsule may be listed. In various embodiments, ingredients in a composition are listed in “weight percent,” (i.e., “wt. %”), based on the total weight of the composition. For example, 100 milligrams of a composition comprising 40 mg A and 60 mg B may be recited as “40 wt. % A and 60 wt. % B, based on the total weight of the composition,” which necessarily totals to “100 wt. %.” The actual weight amounts, (e.g., milligrams or grams) generally refers to amounts added for a particular batch size, (e.g., a batch size of powder usable to fill 100 capsules).

As used herein, the term “dosage form” takes on its ordinary meaning in the pharmaceutical arts as the physical form of a composition designed for a particular administration route. For example, dosage forms include, but are not limited to, injectables, infusible liquids, nasal sprays, nasal gels, topicals such as transdermal creams, ointments and patches, loose powders, tablets, sublingual tabs, capsules, lozenges, syrups, vapors, and so forth. In various embodiments, compositions of interest herein comprise powders, and the dosage form preferably comprises a capsule comprising the powdered composition enclosed, encased or “encapsulated” in the capsule or a tablet comprising the powdered composition compressed into a shape for swallowing or sublingual dissolution.

As used herein, the term “capsule” generally refers to a one or two-piece enclosure for a loose powder, which can be swallowed for oral administration of the powder contents of the capsule, or a soft-shell for a liquid composition, otherwise known as a “gel cap”. In general, “soft” capsules for liquids are one piece, whereas “hard” capsules for powders are two-piece. In some instances, the capsule is said to “encapsulate” the powder contained therein, which can be confusing because the term “encapsulation” is often used, perhaps more correctly, to describe a microscale or nanoscale phenomenon rather than describing something macroscopic like a drug dosage form. In various embodiments, capsules for use herein are hard, stable two-piece shells, or enclosures, capable of stably holding a powder fill, and capable of disintegrating in the gastrointestinal track of an individual. Capsules for use herein may comprise any combination of animal gelatin, plant polysaccharides (carrageenan, cellulosic substances, etc.), or starch or derivatives thereof. In some instances, capsules may further comprise plasticizers, colors, preservatives, disintegrants, lubricants, and various surface treatments such as laser perforations. In various embodiments, a capsule may be transparent so that the contents are visible, or entirely opaque to obscure the contents. In various embodiments, the rate of delivery of a SERM from a two-piece hard capsule having a powder fill may be controlled by any combination of ingredients in the powder fill and ingredients or design configurations of the capsule itself. For example, a capsule intended for immediate release of an active drug may comprise a micronized powder fill in combination with a laser perforated capsule having disintegrants incorporated in the capsule material.

Two-piece hard capsules for use herein can be characterized by a size scale that includes size 5, 4, 3, 2, 1, 0, 0E, 00, 000, 13, 12, 12e1, 11, 10, 7 and Su07, (in increasing order of physical dimensions and internal volume when assembled). Typically, only the capsule sizes from #5 up through about 000 (26.14 mm×9.91 mm, 1.36 mL) would be practical for human oral consumption, and digestive tract (enteral) route of administration. Preferred for use herein are #3 size capsules. A #3 capsule shell weighs about 50 mg if made from vegetarian HPMC and about 48 mg if made from gelatin. As mentioned, and although somewhat more expensive, the vegetarian HPMC shell is preferred because of its faster dissolution rate compared to gelatin. In preferred embodiments, a fill weight for an individual rapid release capsule is from about 100 mg to about 300 mg of loose powder and more preferably from about 120 mg to about 150 mg.

As used herein, the term “subject” or the phrase “a subject in need thereof” refers to any human or non-human animal requiring or desirous of a pharmacological change. For example, a subject in need thereof may be a male human patient clinically diagnosed with low testosterone levels and/or diagnosed with secondary hypogonadotropic hypogonadism. In various embodiments, the subject in need thereof is a person desirous of improved libido or energy as subjectively perceived by that individual. Most importantly, a subject in need thereof can be any human in reasonably good health, but still desirous of heightened sexual experiences or at least less fatigue.

As used herein, the term “treatment” of a subject (e.g., a human) is any type of intervention used in an attempt to alter the natural course of the subject. Treatment includes, but is not limited to, administration of a TD composition in accordance with the present disclosure and may be performed at any time, such as when feeling fatigued or depressed or prior to an engagement with a sexual partner.

As used herein, the term “therapeutically effective amount” refers to a minimum dosage of a composition in accordance with the present disclosure that provides a desired effect. Therefore, a therapeutically effective amount varies by subject, dosage form, concentration of one or more SERMs in the TD composition, and the ultimate results desired. For example, a therapeutically effective amount of a capsule disclosed herein to treat an individual diagnosed with TD and/or secondary hypogonadotropic hypogonadism might be one (1) to two (2) capsules ingested as needed (on an “as needed” basis), wherein each capsule preferably weighs from about 120 mg to about 150 mg and delivers from about 3 mg to 50 mg total active SERM per capsule. In preferred embodiments, each capsule is compounded so as to deliver 3.125 mg, 6.25 mg, 12.5 mg, 25 mg or 50 mg SERM, such as (E)-clomifene (enclomifene).

As used herein, the term “modulate” includes to “increase” or “decrease” one or more quantifiable parameters, optionally by a defined and/or statistically significant amount. By “increase” or “increasing,” “enhance” or “enhancing,” or “stimulate” or “stimulating.” refers generally to the ability of one or more TD compositions in accordance with the present disclosure to produce or cause a greater physiological response (i.e., downstream effects) in a cell or in a subject relative to the response caused by either no TD composition or a control compound. Relevant physiological or cellular responses (in vivo or in vitro) upon administration of a TD composition will be apparent to persons skilled in the art. An “increased” or “enhanced” amount is typically a “statistically significant” amount, and may include an increase that is 1.1, 1.2, 2, 3, 4, 5, 6, 7, 8, 9, 10, 15, 20, 30, 40, 50 or more times (e.g., 500, 1000 times), including all integers and decimal points in between and above 1 (e.g., 1.5, 1.6, 1.7. 1.8), the amount produced by no TD composition (the absence of a bioactive agent) or a control compound.

As used herein, the term “approximately” in reference to amounts refers to plus or minus 5% of the value given, such as wt. %. The term “about,” a in reference to amounts refers to plus or minus 10% of the value given, such as wt. %.

### General Embodiments

In various embodiments, a TD composition in accordance with the present disclosure comprises at least one SERM, optional excipient, and a carrier. In various embodiments, the SERM is selected from the group consisting of tamoxifen, toremifene, raloxifene, lasofoxifene, bazedoxifene, enclomifene, pharmaceutically acceptable salts, solvates or hydrates thereof, and combinations thereof. In various embodiments, the carrier comprises a cellulosic, preferably microcrystalline cellulose. In various embodiments, the TD composition is in the physical form of a loose powder for filling capsules, and wherein the dosage form to administer the composition comprises an immediate release capsule.

In general embodiments of TD compositions, the at least one SERM is present (at a combined weight if more than one) from about 15.0 wt. % to about 25.0 wt. %, based on the total weight of the composition, and depending on the actual SERM chemical species used and the desired strength of the SERM in the finished dosage form. In various embodiments, a SERM may be in the form of a pharmaceutically acceptable salt, such as the citrate salt or other salt to provide a more water soluble and/or absorbable form.

Table 1 sets forth three general embodiments (I, II, III) in accordance with the present disclosure, which vary as to the nature of the SERM and whether a single SERM or combination thereof is used. The compositions are in the physical form of loose powders that can be loaded into capsules to provide dosage forms for oral administration. General embodiments I-III are compositions for use in an immediate release dosage form. In any of the specific compositions under I, II, and III, the loose fill composition may be loaded into two-piece hard shell capsules at a fill level of from about 100 mg to about 300 mg, preferably from about 120 mg to about 150 mg, depending on the nature of the one or more SERM actives. The capsule shell may be selected and/or physically modified as necessary to be more appropriate for immediate decomposition and release.

TABLE 1
General SERM Compositions for the Treatment of TD

| | Compositions | | |
|---|---|---|---|
| Ingredient (wt. %) | I | II | III |
| SERM¹ | 15-18 | 18-22 | 22-25 |
| Carrier² | 82 to 85 | 78 to 82 | 75 to 78 |
| Excipients³ (color, flavor, etc.) | 0 to 5 | 0 to 5 | 0 to 5 |
| Total | 100.00 wt. % | 100.00 wt. % | 100.00 wt. % |
| Physical Appearance | Loose powder for | Loose powder for | Loose powder for |
| | capsule fill | capsule fill | capsule fill |
| Typical Capsule Fill | 100 mg to 300 mg | 100 mg to 300 mg | 100 mg to 300 mg |
| Use | Immediate Release | Immediate Release | Immediate Release |

### Notes to Table 1:

(1) The SERM is selected from the group consisting of tamoxifen, toremifene, raloxifene, lasofoxifene, bazedoxifene, clomifene and mixtures thereof, including pharmaceutically acceptable salts, solvates and hydrates thereof. In preferred compositions, the SERM is enclomifene citrate, and the finished composition is entirely devoid of the other stereoisomer zuclomifene such that the compositions according to TABLE 1 are purposely devoid of estrogenically active substances that are capable of reducing testosterone levels in a male.
(2) In preferred embodiments, the carrier is microcrystalline cellulose (MCC), which also provides a disintegrating effect upon moisture absorption and swelling, negating the need for a separate disintegrant.
(3) Typically the optional excipient includes disintegrant, colors, flavoring agents, sweeteners, preservatives, stabilizers, and so forth. In various embodiments, the flavoring agents are not included, but nonetheless a color, such as a food coloring, may be added for a visual effect when using a transparent and colorless capsule shell. In certain embodiments, the filler is MCC and the need for a separate disintegrant is eliminated.

In various embodiments, a TD composition in accordance with the present disclosure comprises any combination of optional excipients. In various embodiments, a TD composition contains no excipients. In some instances, there is no taste to mask in a swallowed capsule, and perhaps no need for colorants, sweeteners or preservatives. Depending on the SERM, and in view the filled capsule is designed for immediate release, it may be necessary to include a flavorant.

In various embodiments, the optional one or more excipients include any one or combination of flavorant, sweetener, buffer (or acidic agent and/or alkali agent), colorant, disintegrant, intestinal permeation enhancer, stabilizer, preservative, or other pharmaceutically acceptable substance. Any of these materials not specifically mentioned herein may be found in “Handbook of Pharmaceutical Excipients, 6^{th} Edition, R. C Rowe, et al., editors, Pharmaceutical Press, London, 2009, mentioned above in the context of “fillers.” For liquid excipients, or excipients that might be better dispersed if provided in solution, the substance may be sprayed into a ribbon blender with a spray bar as a powdered composition is blending. In this way, a dry blended powder is still obtained, even though small amounts of liquid ingredients are absorbed in homogeneously.

Suitable flavorants can include, for example, flavors, such as, natural flavors, artificial flavors, and combinations thereof. Non-limiting examples of flavor oils include spearmint oil, cinnamon oil, oil of wintergreen (methyl salicylate), peppermint oil, clove oil, bay oil, anise oil, eucalyptus oil, thyme oil, cedar leaf oil, oil of nutmeg, allspice, oil of sage, mace, oil of bitter almonds, and cassia oil. Suitable flavoring agents also include, for example, artificial, natural and synthetic fruit flavors such as vanilla, citrus oils (e.g., lemon, orange, lime, and grapefruit), and fruit essences (e.g., apple, pear, peach, grape, strawberry, raspberry, cherry, plum, pineapple, and apricot), and the like, and combinations thereof.

Other flavorants and fragrant aromatics that may be included individually or in combination include, but are not limited to, anethole, menthol, menthone, menthyl acetate, eucalyptol, borneol, borneol acetate, camphor, 1,8-cineole, cinnamaldehyde, benzaldehyde, citral, thujone, eugenol, limonene, geraniol, citronellol, citronellal, pinene, linalool, thymol, carvone, caryophyllene, linalyl acetate, methyl salicylate, and mixtures thereof. Also, substances that provide scent and flavor include, but are not limited to, 3,3,5-trimethylcyclohexanol, methoxycyclohexanol, benzyl alcohol, anise alcohol, cinnamyl alcohol, β-phenyl ethyl alcohol (2-phenylethanol), cis-3-hexenol, musk xylol, isoeugenol, methyl eugenol, α-amylcinnamic aldehyde, anisaldehyde, n-butyl aldehyde, cumin aldehyde, cyclamen aldehyde, decanal, isobutyl aldehyde, hexyl aldehyde, heptyl aldehyde, n-nonyl aldehyde, nonadienol, hydroxycitronellal, benzaldehyde, methyl nonyl acetaldehyde, dodecanol, α-hexylcinnamic aldehyde, undecenal, heliotropin, vanillin, ethyl vanillin, methyl amyl ketone, methyl β-naphthyl ketone, methyl nonyl ketone, musk ketone, diacetyl, acetyl propionyl, acetyl butyryl, acetophenone, p-methyl acetophenone, ionone, methyl ionone, amyl butyrolactone, diphenyl oxide, methyl phenyl glycidate, γ-nonyl lactone, coumarin, cineole, ethyl methyl phenyl glycidate, methyl formate, isopropyl formate, linalyl formate, ethyl acetate, octyl acetate, methyl acetate, benzyl acetate, butyl propionate, isoamyl acetate, isopropyl isobutyrate, geranyl isovalerate, allyl capronate, butyl heptylate, octyl caprylate octyl, methyl heptynecarboxylate, methine octynecarboxylate, isoacyl caprylate, methyl laurate, ethyl myristate, methyl myristate, ethyl benzoate, benzyl benzoate, methylcarbinylphenyl acetate, isobutyl phenylacetate, methyl cinnamate, cinnamyl cinnamate, ethyl anisate, methyl anthranilate, ethyl pyruvate, ethyl α-butyl butylate, benzyl propionate, butyl acetate, butyl butyrate, p-tert-butylcyclohexyl acetate, cedryl acetate, citronellyl acetate, citronellyl formate, p-cresyl acetate, ethyl butyrate, ethyl caproate, ethyl cinnamate, ethyl phenylacetate, ethylene brassylate, geranyl acetate, geranyl formate, isoamyl salicylate, isoamyl isovalerate, isobornyl acetate, linalyl acetate, methyl anthranilate, methyl dihydrojasmonate, β-phenylethyl acetate, trichloromethylphenyl carbinyl acetate, terpinyl acetate, vetiveryl acetate, and mixtures thereof.

Suitable sweeteners include nutritive carbohydrates such as sucrose, glucose, fructose, glucose, trehalose, galactose, mannitol, sorbitol, xylitol and artificial sweeteners such as saccharin, aspartame, acesulfame K, cyclamates, neotame, sucralose, stevia, and neohesperidin dihydrochalcone (NHDC).

Suitable buffers may comprise one or more acidifying agents or alkaline agents as necessary to neutralize various co-ingredients, form salts of various co-ingredients, and/or achieve a particular pH target for the composition, such as to adjust the local environment in the GI tract as a dosage form dissolves.

Exemplary acidifying agents for use in the present compositions include, but are not limited to, organic acids of any molecular weight and mineral acids (inorganic acids), and mixtures thereof. Organic acids may include mono-carboxylic acids, di-carboxylic acids, or tri-carboxylic acids, and may be saturated or may have any degree of unsaturation. For example, organic acids for use in various embodiments of the composition in accordance to the present disclosure may include, but are not limited to, formic acid, carbonic acid, acetic acid, lactic acid, oxalic acid, propionic acid, valeric acid, enanthic acid, pelargonic acid, butyric acid, lauric acid, docosahexaenoic acid, eicosapentaenoic acid, pyruvic acid, acetoacetic acid, benzoic acid, salicylic acid, aldaric acid, fumaric acid, glutaconic acid, traumatic acid, muconic acid, malonic acid, malic acid, succinic acid, glutaric acid, adipic acid, pimelic acid, suberic acid, azelaic acid, abietic acid, pimaric acid, sebacic acid, phthalic acid, isophthalic acid, terephthalic acid, maleic acid, citric acid, and combinations thereof.

Exemplary alkaline materials include any organic amines, NH₃, alkali metal or alkaline earth hydroxide, any conjugate bases of any organic acids (e.g. R—COO⁻), and any of the salts of carbonic acid, phosphoric acid, nitric acid and sulfuric acid, and any mixtures thereof. For example, alkaline materials for use in various embodiments of the composition in accordance to the present disclosure may include, but are not limited to, NaOH, KOH, NH₃, sodium acetate, sodium succinate, disodium succinate, monosodium citrate, disodium citrate, trisodium citrate, NaH₂PO₄, Na₂HPO₄, Na₃PO₄, KH₂PO₄, K₂HPO₄, K₃PO₄, NaHSO₄, Na₂SO₄, KHSO₄, K₂SO₄, NaHCO₃, NaCO₃, KHCO₃, K₂CO₃, NaH₃P₂O₇, Na₂H₂P₂O₇, Na₃HP₂O₇, Na₄P₂O₇, KH₃P₂O₇, K₂H₂P₂O₇, K₃HP₂O₇, K₄P₂O₇, and mixtures thereof. Any of these chemical species may exist as various hydrates when purchased as raw materials for use in the present compositions.

Exemplary colorants include the pharmaceutically acceptable colors used for capsules and tablet dosage forms, such as the US FDA certified colors, dyes and lakes for use in pharmaceutical capsules, tablets and syrups. These acceptable colorants include the inorganic pigments such as titanium dioxide, yellow iron oxide, red iron oxide and black iron oxide, the organic pigments such as D&C Red 36, Red 30 and Red 34, the solvent soluble colors D&C Yellow 11, Yellow 7, Red 27, Red 21, Red 17, Green 6, and Violet 2, and the water soluble colors D&C Green 8, Yellow 10, Yellow 8, Orange 4, Red 22, Red 28, Red 33, Green 5, quinoline yellow; FD&C Yellow 5, Yellow 6, Red 4, Red 40, Red 3, Green 3, Blue 1, Blue 2, and ponceau 4R, carmoisine, amaranth, patent blue V and black PN, and a number of “organic lakes.”

Suitable disintegrants include, but are not limited to, sodium starch glycolate, croscarmellose sodium, microcrystalline cellulose (MCC), and crospovidone. Added in sufficient quantity, a disintegrant may comprise the entirety of the carrier by weight. For a review of disintegrants that find use in the present compositions, see P. M. Desai, “Review of Disintegrants and the Disintegration Phenomena,” J. Pharm. Sci., 105, 2545-2555 (2016).

Suitable intestinal permeation enhancers include, but are not limited to, surfactants that assist bio-absorption, including, for example, fatty acids and/or esters or salts thereof, bile acids and/or salts thereof. Bile acids/salts and fatty acids and their uses are further described in U.S. Pat. No. 6,287,860, which is incorporated herein in its entirety. In some embodiments, the present disclosure provides combinations of absorption enhancers, for example, fatty acids/salts in combination with bile acids/salts. An exemplary combination is the sodium salt of lauric acid, capric acid and ursodeoxycholic acid (UDCA) for promoting improved intestinal absorption of peptides and other materials. These excipients may be used in the present compositions to assist absorption of the SERM. Additional penetration enhancers include, but are not limited to, polyoxyethylene-9-lauryl ether, polyoxyethylene-20-cetyl ether. For a review of absorption enhancers that find use herein, see B. J. Aungst, “Intestinal Permeation Enhancers,” J. Pharm. Sci., 89(4), 429 (2000).

Stabilizers and preservatives are generally more important for liquid compositions rather than dry powder compositions. Such substances for oral compositions include the parabens, sorbitol, sodium benzoate, benzoic acid, sorbic acid, potassium sorbate, propionic acid, and combinations thereof. Antioxidants include, but are not limited to, vitamin C, vitamin E, butylated hydroxyanisole (BHA), butylated hydroxytoluene (BHT), and propylgallate. For a review see, I. Himoudy, “Preservatives and their role in pharma and clinical research,” International Journal of Pharma Sciences and Scientific Research, 2:4, 134-151 (2016).

### Additional Ingredients for Improved Androgenic Effect

In various embodiments, TD compositions of the present disclosure also include a compound that improves androgenic effects. Preferred are zinc gluconate, zinc picolinate, or ascorbic acid. Any of these compounds can be included at up to about 80 wt. %, such as at levels where any of these compounds can act as filler.

### Extended Release Options

Although ID (instant delivery) dosage forms are preferred, this disclosure should not be viewed as being limited to such. For example, cellulosic substances, such as hydroxypropyl methylcellulose (Hypromellose) may be used to slow delivery of the SERM.

### Exemplary Compositions, Dosage Forms, Methods of Manufacturing and Methods of Administration

Table 2 sets forth exemplary TD compositions in accordance with the present disclosure. Each of the exemplary composition in Table 2 comprise loose powders and each were filled into two-piece hard shell capsules at the fill weights indicated.

TABLE 2
Exemplary SERM Compositions for TD and Dosages Thereof:

| | Compositions | | | | | | | | | | | |
|---|---|---|---|---|---|---|---|---|---|---|---|---|
| Ingredients (wt. %) | 1 | | 2 | | 3 | | 4 | | 5 | | 6 | |
| Enclomifene citrate | 20.0 | | -0- | | -0- | | -0- | | -0- | | -0- | |
| Tamoxifen citrate | -0- | | 20.0 | | -0- | | -0- | | -0- | | -0- | |
| Toremifene citrate | -0- | | -0- | | 20.0 | | -0- | | -0- | | -0- | |
| Raloxifene-HCl | -0- | | -0- | | -0- | | 20.0 | | -0- | | -0- | |
| Lasofoxifene tartrate | -0- | | -0- | | -0- | | -0- | | 20.0 | | -0- | |
| Bazedoxifene acetate | -0- | | -0- | | -0- | | -0- | | -0- | | 20.0 | |
| Microcrystalline Cellulose | 80.0 | | 80.0 | | 80.0 | | 80.0 | | 80.0 | | 80.0 | |
| Total | 100 | wt. % | 100 | wt. % | 100 | wt. % | 100 | wt. % | 100 | wt. % | 100 | wt. % |
| Capsule Fill | 135 | mg | 135 | mg | 135 | mg | 135 | mg | 135 | mg | 135 | mg |
| Active SERM per capsule | 25 | mg | 25 | mg | 25 | mg | 25 | mg | 25 | mg | 25 | mg |

The microcrystalline cellulose MCC used is preferably Avicel® PH-105 (from DuPont®), or Flocel® 101 (from Medisca®).

The compositions are made according to the following procedure: The SERM, such as enclomifene citrate, is first pulverized to a fine powder, and then mixed together with the MCC in a mortar and pestle or Resonant Acoustic Mixer (RAM). The resulting fine powder is then loaded into individual #3 size capsules at the target fill weight.

In various preferred examples, a composition for the treatment of testosterone deficiency consists essentially of enclomifene, a carrier, and optionally an excipient, wherein the composition is entirely devoid of zuclomifene. Such compositions may be filled into individual hard-shell capsules, wherein the hard-shell capsule is either gelatin or the faster dissolving hydroxypropyl methylcellulose, and wherein the hard-shell capsule is perforated with micron-sized holes such as from a laser.

In various embodiments, each capsule encases a loose powder preferably weighing from about 120 mg to about 150 mg, wherein each capsule is configured to deliver from about 3 mg to 50 mg active enclomifene or other SERM. For example, the compositions in TABLE 2 can be adjusted such that the active SERM per capsule is 3.125 mg, 6.25 mg, 12.5 mg. 25 mg, or 50 mg.

### Dosage Regimens

In various embodiments of, methods of treating testosterone deficiency (TD) and/or secondary hypogonadotropic hypogonadism, or symptoms manifesting therefrom, are described.

In general, a method of treating testosterone deficiency (TD) and/or secondary hypogonadotropic hypogonadism in an individual in need thereof comprises orally administering to the individual a therapeutically effective amount of a composition comprising at least one SERM. In various embodiments, the individual in need thereof has been diagnosed as having testosterone deficiency or secondary hypogonadotropic hypogonadism or is showing associated symptoms such as reduced sex drive, erectile dysfunction, loss of body hair, reduced beard growth, muscle atrophy, fatigue, or depression.

In various embodiments, a therapeutically effective amount is from about 3 mg to about 50 mg total active SERM compounds. In preferred embodiments, a therapeutically effective amount is from about 3 mg to about 50 mg total active enclomifene. In certain examples, a therapeutically effective amount is 3.125 mg. 6.25 mg, 12.5 mg. 25 mg and 50 mg enclomifene per day. Dosage may be a single capsule per day, or a combination of two capsules per day such as to achieve a dosage between these fixed amounts. For example, an individual may require 37.5 mg enclomifene per day, and thus may take one 25 mg capsule and one 12.5 mg capsule at the same time per day.

In various embodiments, a method of treating reduced sex drive, erectile dysfunction, loss of body hair, reduced beard growth, muscle atrophy, fatigue, or depression in a male subject in need thereof comprises administering a therapeutically effective amount of a TD composition comprising at least one SERM as needed. In various aspects, this on-demand or as needed administration replaces the chronic daily dosage regimen currently prescribed for SERM-based treatments of TD.

TD compositions of the present disclosure are delivered orally, packed as dry loose powders into capsules for swallowing. In various embodiments, the TD compositions comprise dry blended loose powders, with the dosage form comprising a hard-shell capsule comprising the dry blended loose powder contained therein. In preferred embodiments, the capsules are fast dissolving, such as comprising HPMC, and the capsules are optionally laser perforated to accelerate entrainment of gastric juices upon ingestion.

Compositions for the treatment of testosterone deficiency (TD) in male subjects and methods thereof are provided. In the detailed description herein, references to “various embodiments”, “one embodiment”, “an embodiment”, “an example embodiment”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Benefits, other advantages, and solutions to problems have been described herein with regard to specific embodiments. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to ‘at least one of A, B, and C’ or ‘at least one of A, B, or C’ is used in the claims or specification, it is intended that the phrase be interpreted to mean that A alone may be present in an embodiment, B alone may be present in an embodiment, C alone may be present in an embodiment, or that any combination of the elements A, B and C may be present in a single embodiment: for example, A and B, A and C, B and C, or A and B and C.

All structural, chemical, and functional equivalents to the elements of the above-described various embodiments that are known to those of ordinary skill in the art are expressly incorporated herein by reference and are intended to be encompassed by the present claims. Moreover, it is not necessary for a composition or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims. No claim element is intended to invoke 35 U.S.C. 112(f) unless the element is expressly recited using the phrase “means for.” As used herein, the terms “comprises,” “comprising,” or any other variation thereof, are intended to cover a non-exclusive inclusion, such that a chemical, chemical composition, process, method, article, or apparatus that comprises a list of elements does not include only those elements but may include other elements not expressly listed or inherent to such chemical, chemical composition, process, method, article, or apparatus.

1. A pharmaceutical composition for treatment of testosterone deficiency (TD) and/or secondary hypogonadotropic hypogonadism, said composition comprising:
at least one selective estrogen receptor modulator (SERM);
a carrier; and
optionally, an excipient;
wherein the composition is in the form of a loose dry powder amenable to packaging into individual dosage forms, and
wherein the composition is entirely devoid of estrogenically active substances.

2. The pharmaceutical composition of claim 1, wherein the selective estrogen receptor modulator (SERM) is selected from the group consisting of tamoxifen, toremifene, raloxifene, lasofoxifene, bazedoxifene, enclomifene, pharmaceutically acceptable salts thereof, and mixtures thereof.

3. A pharmaceutical dosage form for oral administration comprising:
a capsule; and
a composition according to claim 1 enclosed therein.

4. The pharmaceutical composition of claim 1, wherein the carrier comprises microcrystalline cellulose.

5. The pharmaceutical composition of claim 1, wherein the optional excipient is at least one of a disintegrant, a colorant, a flavoring agent, a sweetener, a preservative, and a stabilizer.

6. The pharmaceutical composition of claim 1 consisting essentially of enclomifene ((E)-2-(4-(2-chloro-1,2-diphenylvinyl)phenoxy)-N,N-diethylethan-1-amine) and microcrystalline cellulose, wherein the pharmaceutical composition is entirely devoid of zuclomifene ((Z)-2-(4-(2-chloro-1,2-diphenylvinyl)phenoxy)-N,N-diethylethan-1-amine).

7. A single dose pharmaceutical capsule consisting essentially of:
from about 100 mg to about 300 mg of a composition consisting essentially of from about 15 wt. % to about 25 wt. % enclomifene citrate and from about 75 wt. % to about 85 wt. % microcrystalline cellulose, based on the total weight of the composition; and
a capsule enclosing said composition,
wherein said composition is entirely devoid of zuclomifene ((Z)-2-(4-(2-chloro-1,2-diphenylvinyl)phenoxy)-N,N-diethylethan-1-amine), or salts, solvates, hydrates, or prodrugs thereof.

8. The single dose pharmaceutical capsule of claim 7, wherein the capsule comprises gelatin or vegetarian hydroxypropyl methylcellulose (HPMC), and wherein the capsule is optionally laser-perforated with a plurality of holes for water ingress.

9. A method of treating testosterone deficiency (TD) and/or secondary hypogonadotropic hypogonadism in a male individual, the method comprising orally administering to the individual in need thereof a therapeutically effective amount of a composition comprising at least one selective estrogen receptor modulator (SERM); a carrier; and optionally, an excipient, wherein the composition is entirely devoid of estrogenically active substances.

10. The method of claim 9, wherein the testosterone deficiency (TD) and/or secondary hypogonadotropic hypogonadism is characterized by reduced sex drive, erectile dysfunction, loss of body hair, reduced beard growth, muscle atrophy, fatigue, or depression.

11. The method of claim 9, wherein the testosterone deficiency (TD) and/or secondary hypogonadotropic hypogonadism is characterized by testicular atrophy caused by injectable testosterone replacement therapy (TRT).

12. The method of claim 9, wherein the therapeutically effective amount comprises orally administering from about 3 mg to about 50 mg of the at least one SERM daily.

13. The method of claim 9, wherein the at least one SERM is enclomifene ((E)-2-(4-(2-chloro-1,2-diphenylvinyl)phenoxy)-N,N-diethylethan-1-amine) and the carrier is microcrystalline cellulose.

14. The method of claim 13, wherein the therapeutically effective amount comprises daily administration of a capsule enclosing said composition, wherein each capsule comprises from about 120 mg to about 150 mg of the composition, and wherein each capsule delivers from about 3 mg to about 50 mg enclomifene actives.

15. The method of claim 14, wherein each capsule delivers about 3.125 mg, 6.25 mg, 12.5 mg, 25 mg or 50 mg enclomifene actives.
